(19) United States
(12) Patent Application Publication
(10) Pub. No.: US 2002/0132267 A1
(43) Pub. Date: Sep. 19, 2002

(54) Combination drug test and adulterant test device

(76) Inventor: Raphael C. Wong, Irvine, CA (US)

Correspondence Address: Vic Y. Lin, MYERS DAWES & ANDRAS LLP, Suite 1150, 19900 MacArthur Blvd., Irvine, CA, 92612, US

(21) Appl. No.: 09/810,783

(22) Filed: Mar. 16, 2001

Related U.S. Application Data
(60) Provisional application No. 60/219,921, filed on Jul. 21, 2000.

(51) Int. Cl. G01N 33/53; C12M 1/34

(52) U.S. Cl. 435/7.1; 435/287.2

(57) ABSTRACT

A combination drug test and adulterant test device comprises a container having an adulterant test strip and a drug test strip. The strips are held in place and kept physically and fluidly separate from each other by a combination of recesses, ribs, and uneven surfaces. Apertures are formed in the upper portion of the body for providing access to the strips to enable portions of the sample to be disposed. Windows are formed in the upper portion for viewing the indicator portions of the drug test strips. A method for manufacturing drug test strips is also provided.

### RELATED APPLICATIONS

This application relates to and claims priority from U.S. Provisional Application No. 60/219,921 entitled COMBINATION DRUG TEST AND ADULTERANT TEST DEVICE filed on Jul. 21, 2000, the disclosure of which is herein incorporated by reference as if fully set forth.

### BACKGROUND OF THE INVENTION

1. Field of the Invention

The invention relates generally to chemical testing devices for fluids.

2. Description of Related Art

As drug screening becomes increasingly prevalent, many of those being tested have resorted to certain substances to nullify a drug test by, for instance, making an otherwise positive result appear as negative. Such substances include chemicals added to the fluid sample, such as bleach, as well as chemicals to be ingested prior to producing the sample. Though several devices and methods are known in the art for testing samples for the presence of illegal drugs, much less art exists for testing of any adulteration in the sample. Furthermore, conventional adulteration testing devices are both physically and procedurally separate from drug testing devices. Thus, a technician who desires to test a sample for both adulteration and drug use must do so with separate devices.

Using separate devices leads to greater complication for a variety of reasons. Since technicians may often deal with a plurality of samples at once, samples may get mixed up in the process as they are being transported from one device to another. Such confusion would nullify not only one result, but an entire group of test results, thus requiring each sample to be retested. Having to use multiple devices also leads to a great deal of labor and time as a high number of steps are involved. Furthermore, having to dispose portions of samples in multiple devices also leads to a high probability of spillage which, in the case of urine samples, can lead to sanitation problems.

A common adulteration testing device is a strip that is dipped into a sample, which strip is also known as a dip stick. Since chemicals from the dipstick may leach out into the sample, such an adulteration test will negate the remainder of the sample for any further testing. Even if the effect of such chemicals is negligible with respect to a drug test, a donor will have legal grounds to challenge the results of any drug test which involved a sample that had previously been in contact with an adulteration dip stick. Therefore, if an adulteration test is desired, technicians are required to split a sample into multiple portions held in separate compartments so that one portion may be tested for adulteration, while another untouched portion may be tested for drugs. Such splitting incurs greater costs as more materials and labor is required.

Furthermore, typical drug testing devices employ a chromograph strip that involves lateral flow technology whereas typical adulteration test devices employ a dip-and-read mechanism that involves a chemical change upon direct contact. Therefore, the development of a multi-testing device has not occurred due to the technological differences in testing for an adulterant versus testing for a drug.

### SUMMARY OF THE INVENTION

In one aspect, the drug testing device is provided for testing for both adulterants and drugs in a fluid sample. The device comprises a main body having a lower portion, or base, and an upper portion, or cover. The upper portion and lower portion of the main body may be integral or separate. An adulterant test strip is disposed in a first area of the lower portion of the main body. A first aperture defined in the upper portion of the main body above the first area is open to the adulterant test strip. A drug test strip is disposed in a second area of the lower portion of the main body that is separate from the first area. A second aperture defined in the upper portion of the main body above the second area is open to the drug test strip. A first plurality of protrusions on a top surface of the lower portion of the main body holds the adulterant test strip in a fixed position while a second plurality of protrusions on the top surface of the lower portion of the main body holds the drug test strip in a fixed position.

The device further comprises a rim on a lower surface of the top portion of the main body that surrounds the second aperture. The first plurality of protrusions and the second plurality of protrusions block fluid communication between the adulterant test strip and the drug test strip. The second aperture is disposed over an initial absorption portion of the drug test strip. A third aperture is defined in the upper portion of the main body and disposed over an indicator portion of the drug test strip. The lower portion comprises a first recess in the first area for receiving the adulterant test strip and a second recess in the second area for receiving the adulterant test strip.

In another aspect, an apparatus for testing the presence of both drugs and adulterants comprises a first region and a second region separate from the first region. An adulterant test strip is disposed in the first region, and a drug test strip is disposed in the second region. A first aperture is disposed in the first region and open to the adulterant test strip. A second aperture is disposed in the second region and open to the drug test strip. The first region comprises a first space for receiving the adulterant test strip and a first plurality of dividers holding the drug test strip and preventing fluid communication between the drug test strip and the adulterant test strip. The second region comprises a second space for receiving the drug test strip, and a second plurality of dividers holding the drug test strip and preventing fluid communication between the drug test strip and the adulterant test strip. The first region is kept physically and fluidly separate from the second region by a separator. The separator may comprise a recessed floor in the first region, a raised floor in the second region, and/or a barrier disposed between the first and second region.

The apparatus further comprises a third aperture disposed in the second region that is open to an indicator portion of the drug test strip whereas the second aperture is open to an initial absorption portion of the drug test strip.

An adulteration test strip according to the invention is also provided employing a lateral flow absorption process as opposed to the direct contact process of conventional adulterant testing means. The lateral flow adulterant test strip comprises a backing, an absorption pad disposed adjacent to the backing, and an adulteration test pad adapted for testing the presence of an adulterant in a fluid sample. The adulteration test pad is disposed adjacent to the absorption pad. The absorption pad has a near end and a far end that defines a length smaller than a length of the backing. The adulteration test pad is disposed adjacent to the far end of the absorption pad. A near end of the absorption pad is substantially flush with a near end of the backing.

A method for manufacturing a combination drug and adulterant testing device is also provided. The method comprises: providing a main body having at least a first region and a second region; disposing an adulterant test strip in the first region; disposing a drug test strip in the second region; separating the adulterant test strip from the drug test strip to prevent any fluid communication therebetween; providing access to the adulterant test strip; and providing access to the drug test strip.

Providing a main body having at least a first region and a second region comprises providing a base and a cover. Separating the adulterant test strip from the drug test strip to prevent any fluid communication therebetween comprises: forming a first compartment for receiving the adulterant test strip; and forming a separate second compartment for receiving the drug test strip. Forming a first compartment for receiving the adulterant test strip comprises forming a first recess and a first plurality of protrusions surrounding the first recess. Forming a second compartment for receiving the drug test strip comprises forming a second recess and a second plurality of protrusions surrounding the second recess. Disposing an adulterant test strip in the first region comprises disposing the adulterant test strip in the first compartment. Disposing a drug test strip in the second region comprises disposing the drug test strip in the second compartment. Providing access to the adulterant test strip comprises forming a first aperture in the cover that is open to the adulterant test strip. Providing access to the drug test strip comprises forming a second aperture in the cover that is open to the drug test strip. The method further comprises forming a third aperture that is open to an indicator portion of the drug test strip.

In conclusion, a combination drug test and adulterant test device comprises a container having an adulterant test strip and a drug test strip. The strips are held in place and kept physically and fluidly separate from each other by a combination of recesses, ribs, and uneven surfaces. Apertures are formed in the upper portion of the body for providing access to the strips to enable portions of the sample to be disposed. Windows are formed in the upper portion for viewing the indicator portions of the drug test strips. A method for manufacturing drug test strips is also provided.

The invention, now having been briefly summarized, may be better visualized by turning to the following drawings wherein like elements are referenced by like numerals.

### BRIEF DESCRIPTION OF THE DRAWINGS

FIG. 1 is an exploded view of a combination adulterant and drug test device according to the invention;

FIG. 2 is a perspective view of the test device;

FIG. 3 is a top plan view of the base of the test device with test strips removed for clarity;

FIG. 4 is a top plan view of the base of the test device with test strips disposed;

FIG. 5 is a bottom plan view of the cover of the test device;

FIG. 6 is a top plan view of the test device;

FIG. 7 is an axial cross-sectional view of the test device taken along lines 7′-7′ of FIG. 6;

FIG. 8 is a transverse cross-sectional view of the test device taken along lines 8′-8′ of FIG. 6;

FIG. 9 is a transverse cross-sectional view of the test device taken along lines 9′-9′ of FIG. 6;

FIG. 10 is an exploded perspective view of an alternate embodiment of the test device;

FIG. 11 is a perspective view of an adulterant test strip employed in the alternate embodiment of FIG. 10.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS

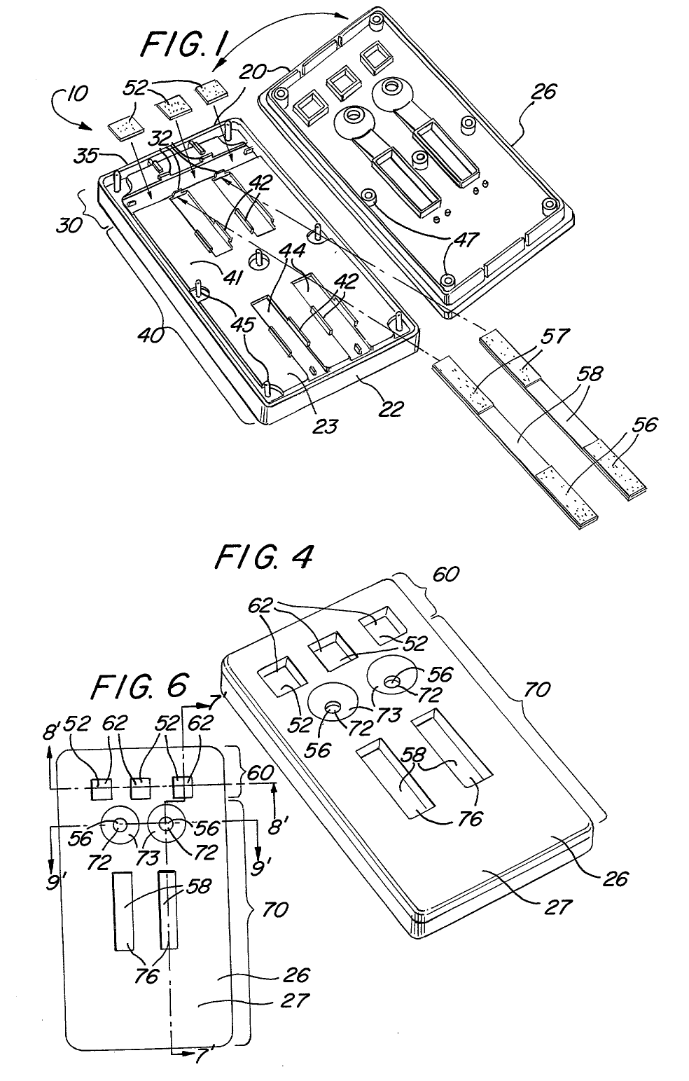

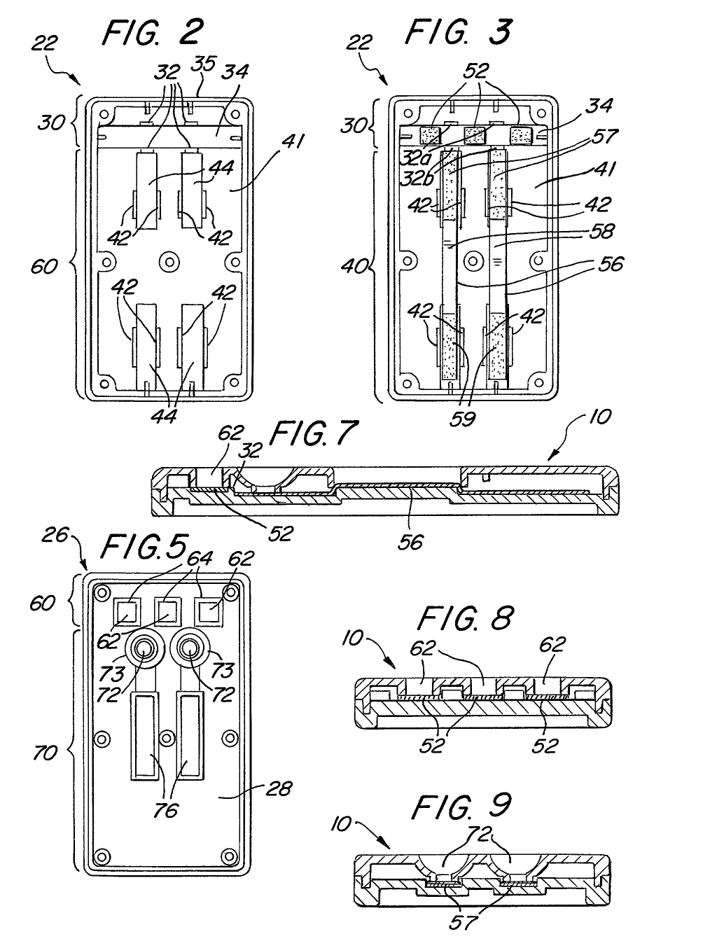

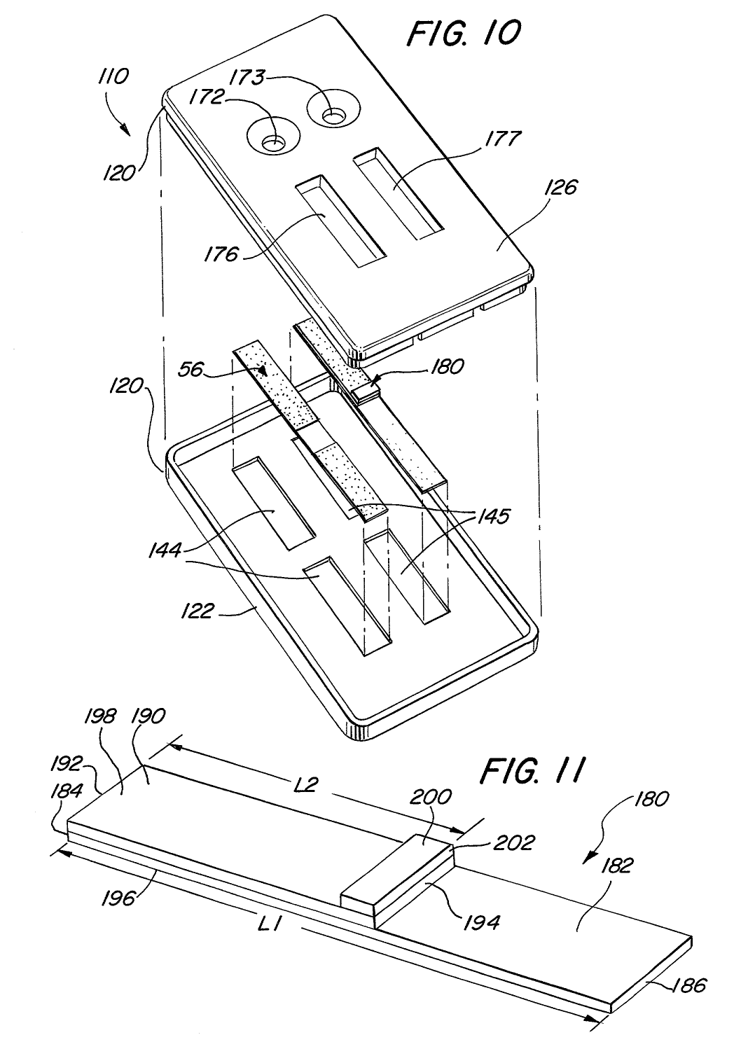

FIG. 1 is an exploded view of a fluid testing apparatus 10 according to the present invention. The apparatus 10 comprises a main body 20 having a lower portion, or base, 22, and an upper portion, or cover 26. It is to be understood that the main body 20 may comprise a unitary structure wherein the lower portion 22 would be integral with the upper portion 26, or a modular structure wherein the lower portion 22 would be separate from the upper portion 26, as shown in the preferred embodiment in FIG. 1. In a preferred embodiment, the main body 20 comprises a cassette. Fasteners 45 and sleeves 47 may be employed for coupling the base 22 and cover 26 together.

In FIGS. 1-3, the base 22 comprises a first, or adulterant, region 30 configured for receiving adulterant test strips 52, and a second, or drug test, region 40 configured for receiving drug test strips 56. A first plurality of protrusions, or ribs, 32 are formed on the upper surface 23 of the base 22 in the first region, or first area, 30. The protrusions 32 serve not only to hold the adulterant test strips 52 in place, but also to keep adulterant test strips 52 spaced apart and fluidly separate from the drug test strips 56 so as to prevent any fluid communication between the adulterant test strips 52 and the drug test strips 56. In FIG. 3, the protrusions, or barriers, 32b disposed between the first region 30 and the second region 40 block any fluid communication between the adulterant strips 52 and the drug test strips 56. A recessed floor 34 provided in the first region 30 for receiving the adulterant test strips 52 works to further separate the strips 52, 56. Alternatively stated, a raised floor 41 in the second region separates the strips 52, 56. Thus, the barriers 32b and uneven floors 34, 41 serve as separators of the adulterant test strips 52 and the drug test strips 56. Optional cavities and additional ribs may be provided in the recessed floor for separating the adulterant test strips 52 from one another. As an example and not by way of limitation, the adulterant test strips 52 may test for different adulterants, such as bleach, creatinine, nitrite, glutaraldehyde, pyridinium cholorochromate. The adulterant test strips 52 may also indicate the pH and specific gravity of the sample. Since the adulterant test strips 52 need not be elongate, the adulterant test strips 52 may be grouped together in a smaller area than that which is necessary for the drug test strips 56. In a preferred embodiment, the group of adulterant test strips 52 are disposed adjacent to a first end 35 of the base 22.

The second region, or second area, 40 of the base 22 includes a raised surface 41 as compared to the recessed floor 34 of the first region 30. In the second region 40, a plurality of recesses, or cavities, 44 are formed in the top surface 23 for receiving drug test strips 56. A plurality of protrusions, or ribs, 42 border the recesses 44 so as to hold the drug test strips 56 in place. The recesses 44 and protrusions 42 form compartments, or spaces, for receiving the drug test strips 56. In a preferred embodiment, the drug test strip 56 comprises a chromograph strip having antibodies disposed at the near, or initial absorption, portion 57 where fluid initially comes into contact. The antibodies react with either antigens disposed on the strip 56, in which case a visible band appears to indicate a negative test result, or with antigens in the fluid, in which case the absence of a band indicates a positive test result. The strip 56 includes an indicator portion, or mid-portion, 58 where the visible bands, or lack thereof, would appear. In a preferred embodiment, the near and far portions 57, 59 are disposed in the recesses 44 of the second region 40.

Thus, it will be appreciated that the combination of the protrusions, recesses, and uneven surfaces of the first and second regions 30, 40, respectively, keep the adulterant test strips 52 separate from the drug test strips 56 and prevent any fluid communication therebetween. The importance of keeping the two types of strips separate lies in the possibility that certain chemicals in one type of test strip may leach and contaminate the results indicated in the other type of test strip.

In FIGS. 4-6, the cover 26 includes a top surface 27 and a bottom surface 28. The cover 26 further comprises a first, or adulterant region, 60, and a second, or drug test region, 70 corresponding to the first and second regions 30, 40, respectively, of the base 20. A first plurality of apertures, or adulterant apertures, 62 are disposed in the first region 60 above the adulterant test strips 52. The adulterant apertures 62 are open to the adulterant test strips 52 thus providing access thereto. On the bottom surface 28 of the cover 26, rims 64 surround each adulterant aperture 62. The rims 64 help prevent fluid disposed in the adulterant aperture 62 from escaping by sealing the edges of the adulterant test strips 52.

Absorption apertures, or drug testing apertures, 72 and indicator apertures, or windows, 76 are disposed in the second region 70 of the cover 26. The absorption apertures 72 are disposed above and open to the initial absorption portions 57 of the drug test strips 56. In a preferred embodiment, the surrounding region 73 of the absorption apertures 72 is conical to provide a well for receiving drops of the fluid. In a preferred embodiment, the adulterant apertures 62 are shaped differently from the absorption apertures 72 to visually notify a user of the difference in functions. The indicator apertures 76 are disposed above and open to the indicator portions 58 of the drug test strips 56.

With the structure of the apparatus 10 having been described, turn now to its operation. In FIGS. 7-9, a user disposes portions of the fluid sample into the adulterant apertures 62 and the absorption apertures 72. Though the user may apply the sample to the apertures 62, 72 in any sequence, it is preferable to apply the sample to the adulterant apertures 62 first in order to determine if the sample has been contaminated with adulterants. Nonetheless, a user may dispose the sample into the absorption apertures 72 regardless of the results of the adulterant strips 52. The adulterant strips 52 absorb the sample disposed in the adulterant apertures 62. If the sample contains the adulterant for which the adulterant strip 52 recognizes, the adulterants strip 52 will turn to a specific color to indicate the presence of the adulterant. The initial absorption portion 57 of each drug test strip 56 absorbs the sample disposed in the absorption apertures 72. The sample travels along the drug test strip 56 and causes indicator lines to show in the indicator portion 58 if certain chemicals, such as illegal drugs, are present in the sample.

FIGS. 10 and 11 illustrate an alternate embodiment of a combination testing device 110. In FIG. 10, a main body 120 comprises a base 122 and cover 126. A first absorption aperture 172 and a second absorption aperture 173 is defined in the cover 126. A first indicator aperture 176 and a second indicator aperture 177 are disposed adjacent to the absorption apertures 172, 173, respectively. In the base 122, a first group of recesses 144 is defined for holding a drug test strip 56. The drug test strip 56 may be substantially similar to the drug test strip 56 described above in connection with FIGS. 1-9. A second group of recesses 145 is defined in the base 122 and adapted for holding a lateral flow, adulteration test strip 180 according to the invention. The first indicator aperture 176 is open to the drug test strip 56 so that the results may be viewed. Likewise, the second indicator aperture 177 is open to the adulteration test strip 180, and more specifically to the adulteration test pad 200 described below, such that the results its result may be displayed.

FIG. 11 is a perspective view of the lateral flow, adulteration test strip 180. The adulteration test strip 180 comprises a backing 182 having a first, near end 184 and a second, far end 186. The near end 184 and the far end 186 define a length “L1” of the backing 182. The adulteration test strip 180 includes a lateral flow absorption pad 190 disposed adjacent to the backing 182. The absorption pad 190 also comprises a first, near end 192, a second, far end 194 that define a length “L2” that is smaller than the length “L1” of the backing 182. The absorption pad 190 also comprises a bottom side 196 and a top side 198. The absorption pad 190 operates in the same manner as a chromograph strip in that fluid initially absorbed at the near end 192 travels along the pad 190 to the far end 194.

An adulteration test pad 200 is disposed on the top side 190 of the absorption pad 190 and preferably adjacent to the second, far end 194. The adulteration test pad 200 may be composed of substantially the same materials and chemicals as the adulteration test strip 52 described above in connection with FIGS. 1-9.

In FIGS. 11 and 12, the adulteration test strip 180 is received in the second group of recesses 145. The second absorption aperture 173 is open to the absorption pad 190 adjacent the near end 192. The second indicator aperture 177 is open to the adulteration test pad 200. Thus, droplets of sample fluid disposed in the second absorption aperture 173 will contact the absorption pad 190 and travel from the near end 192 to the far end 194. As the fluid flows laterally to the far end 194, it will contact the adulteration test pad 200. Depending upon the presence of any adulterants in the sample, the adulteration test pad 200 may change color to indicate the presence thereof. Since color may leech from the adulteration pad 200 into the absorption pad 190, the adulteration pad 200 is preferably disposed at the far end 194 of the absorption pad 190 such that the fluid traveling along the absorption pad 190 cannot carry any colors of the adulteration pad 200 beyond its location. Thus, a far end 202 of the adulteration test pad 200 is preferably flush with the far end 194 of the absorption pad 190. In a preferred embodiment of the adulteration test strip 180, the near end 192 of the absorption pad 190 is substantially flush with the near end 184 of the backing 182.

In the alternative embodiment shown in FIGS. 10 and 11, it will be appreciated that the lateral flow technology of conventional drug test strips is now incorporated into an adulteration test strip.

It will be appreciated that the testing of both adulterants and drugs can be conveniently accomplished in one compact device. Technicians can easily test for the presence of adulterants and certain drugs in a fluid sample without having to transport the sample from one device to another. Furthermore, having the adulterant test result and drug test result shown in a single apparatus avoids any possible mix-ups that would otherwise exist with multiple devices.

A method is also provided for manufacturing a combination drug and adulterant testing device. A main body is provided having at least a first region and a second region. An adulterant test strip is disposed in the first region. A drug test strip is disposed in the second region and separated from the drug test strip to prevent any fluid communication therebetween. Access is provided to the adulterant test strip via adulterant apertures and to the drug test strip via absorption apertures.

Providing a main body may include providing a base and a cover which are coupled together, such as by press fitting. To separate the adulterant test strip from the drug test strip, and thereby to prevent fluid communication between therebetween, a first compartment is formed for receiving the adulterant test strip, and a separate second compartment is formed for receiving the drug test strip. To form a first compartment for receiving the adulterant test strip, a first recess may be formed and surrounded by a first plurality of protrusions. Forming a second compartment for receiving the drug test strip comprises forming a second recess and a second plurality of protrusions surrounding the second recess. Disposing an adulterant test strip in the first region comprises disposing the adulterant test strip in the first compartment. Disposing a drug test strip in the second region comprises disposing the drug test strip in the second compartment. A third aperture is formed that is open to the indicator portion of the drug test strip.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. The claims are thus to be understood to include what is specifically illustrated and described above, what is conceptionally equivalent, what can be obviously substituted and also what incorporates the essential idea of the invention.

What is claimed is:

1. A drug testing device comprising:
a main body having a lower portion and an upper portion;
an adulterant test strip disposed in a first area of the lower portion of the main body;
a drug test strip disposed in a second area of the lower portion of the main body that is separate from the first area;
a first aperture defined in the upper portion of the main body above the first area; and
a second aperture defined in the upper portion of the main body above the second area.

2. The device of claim 1 further comprising:
a first plurality of protrusions on a top surface of the lower portion of the main body adjacent to the adulterant test strip; and
a second plurality of protrusions on the top surface of the lower portion of the main body adjacent to the drug test strip.

3. The device of claim 2 further comprising a rim on a lower surface of the top portion of the main body, the rim surrounding the second aperture.

4. The device of claim 2 wherein the first plurality of protrusions block fluid communication between the adulterant test strip and the drug test strip.

5. The device of claim 1 wherein the second aperture is disposed over an initial absorption portion of the drug test strip, the device further comprising a third aperture defined in the upper portion of the main body and disposed over an indicator portion of the drug test strip.

6. The device of claim 1 wherein the upper portion and lower portion of the main body are integral.

7. The device of claim 1 wherein the upper portion and lower portion of the main body are separate.

8. The device of claim 1 wherein the lower portion comprises a recessed floor in the first area for receiving the adulterant test strip and a recess in the second area for receiving the adulterant test strip.

9. The device of claim 1 wherein the adulterant test strip comprises a backing, an absorption pad disposed on the backing, and an adulteration test pad disposed on the absorption pad.

10. An apparatus for testing the presence of both drugs and adulterants, the apparatus comprising:
a first region;
a second region separate from the first region;
an adulterant test strip disposed in the first region;
a drug test strip disposed in the second region;
a first aperture disposed in the first region and open to the adulterant test strip; and
a second aperture disposed in the second region and open to the drug test strip.

11. The apparatus of claim 10 wherein the first region comprises:
a first space for receiving the adulterant test strip; and
a first plurality of dividers preventing fluid communication between the drug test strip and the adulterant test strip.

12. The apparatus of claim 11 wherein the second region comprises:
a second space for receiving the drug test strip; and
a second plurality of dividers holding the drug test strip in place.

13. The apparatus of claim 10 further comprising a third aperture disposed in the second region, wherein:
the second aperture is open to an initial absorption portion of the drug test strip; and
the third aperture is open to an indicator portion of the drug test strip.

14. The apparatus of claim 10 further comprising a separator separating the first region from the second region.

15. The apparatus of claim 14 wherein the separator comprises a recessed floor in the first region.

16. The apparatus of claim 14 wherein the separator comprises a raised floor in the second region.

17. The apparatus of claim 14 wherein the separator comprises a barrier disposed between the first region and the second region.

18. The apparatus of claim 10 wherein the adulterant test strip comprises a backing, an absorption pad disposed on the backing, and an adulteration test pad disposed on the absorption pad.

19. A lateral flow adulterant test strip comprising:
a backing;
an absorption pad disposed adjacent to the backing, the absorption pad having a near end and a far end; and
an adulteration test pad adapted for testing the presence of an adulterant in a fluid sample, the adulteration test pad being disposed adjacent to the absorption pad.

20. The adulteration test strip of claim 19 wherein the absorption pad has a length smaller than a length of the backing.

21. The adulteration test strip of claim 20 wherein the adulteration test pad is disposed adjacent to the far end of the absorption pad.

22. The adulteration test strip of claim 21 wherein a near end of the absorption pad is substantially flush with a near end of the backing.

23. A method for manufacturing a combination drug and adulterant testing device, the method comprising:
providing a main body having at least a first region and a second region;
disposing an adulterant test strip in the first region;
disposing a drug test strip in the second region;
separating the adulterant test strip from the drug test strip to prevent any fluid communication therebetween;
providing access to the adulterant test strip; and
providing access to the drug test strip.

24. The method of claim 23 wherein providing a main body having at least a first region and a second region comprises providing a base and a cover.

25. The method of claim 24 wherein separating the adulterant test strip from the drug test strip to prevent any fluid communication therebetween comprises:
forming a first compartment for receiving the adulterant test strip; and
forming a separate second compartment for receiving the drug test strip.

26. The method of claim 25 wherein:
forming a first compartment for receiving the adulterant test strip comprises forming a recessed floor and a first plurality of protrusions surrounding the recessed floor; and
forming a second compartment for receiving the drug test strip comprises forming a recess and a second plurality of protrusions surrounding the recess.

27. The method of claim 26 wherein:
disposing an adulterant test strip in the first region comprises disposing the adulterant test strip in the first compartment; and
disposing a drug test strip in the second region comprises disposing the drug test strip in the second compartment.

28. The method of claim 24 wherein:
providing access to the adulterant test strip comprises forming a first aperture in the cover that is open to the adulterant test strip; and
providing access to the drug test strip comprises forming a second aperture in the cover that is open to the drug test strip.

29. The method of claim 28 further comprising forming a third aperture that is open to an indicator portion of the drug test strip.
